(19) United States
(12) Patent Application Publication
(10) Pub. No.: US 2007/0213324 A1
(43) Pub. Date: Sep. 13, 2007

(54) Stable pharmaceutical composition of carisoprodol and meloxicam

(75) Inventors: Antonio Santiago Regalado, Col. Lomas Estrella (MX); Sixto Serafin Espinoza Leon, Col. Los Sauces (MX)

Correspondence Address: REINHART BOERNER VAN DEUREN P.C., 2215 PERRYGREEN WAY, ROCKFORD, IL, 61107, US

(73) Assignee: LABORATORIOS SILANES S.A. DE C.V., Colonia Del Valle (MX)

(21) Appl. No.: 11/655,517

(22) Filed: Jan. 19, 2007

(30) Foreign Application Priority Data
Jan. 20, 2006 (MX) PA/A/2006/000760

(51) Int. Cl. A61K 31/541; A61K 31/138

(52) U.S. Cl. 514/226.5; 514/651

(57) ABSTRACT

The present invention provides a pharmaceutical composition or formulation in the form of tablets of proved stability and describes the obtainment process. This formulation combines the therapeutic action of meloxicam as an anti-inflammatory and carisoprodol as and analgesic and anti-inflammatory. Administering the two components jointly produces beneficial results which are not seen when the two active components are administered separately.

### CROSS-REFERENCE TO RELATED PATENT APPLICATIONS

This patent application claims priority to Mexican Patent Application No. PA/a/2006/000760 filed Jan. 20, 2006, the teachings and disclosure of which are hereby incorporated in their entireties by reference thereto.

### TECHNICAL FIELD OF THE INVENTION

The present invention consists of providing a pharmaceutical composition in form of tablets of proven stability. In addition, the present invention describes how to obtain this pharmaceutical composition. The pharmaceutical composition combines the therapeutic action of Carisoprodol and Meloxicam which produce a synergic effect and as a result are more effective in controlling pain. In addition certain side effects are reduced using medication with Carisoprodol and Meloxicam together.

### BACKGROUND OF THE INVENTION

The present invention proposes a new stable pharmaceutical composition designed to take advantage of the benefits of both of its components: Meloxicam and Carisoprodol, in a single form of dosage. The use of both pharmaceuticals jointly has no precedent in prior art. The studies carried out by our team of researchers have demonstrated the benefits of using both components together. Unexpectedly, the investigators have found that some benefits to the patients are greater when the components are used as a medication jointly than separately.

The invention is based upon the results obtained from phase I and II clinical studies. These studies correspond to the assessment of bioavailability, safety, effectiveness, and possible side effects.

The closest precursor to the invention corresponds to the use of the components independently or separately, as described below:

Meloxicam: Meloxicam, an oxicam derivative, is a member of the nonsteroidal anti-inflammatory family of drugs. Meloxicam is indicated to treat the signs and symptoms of osteoarthritis and is also used as an analgesic to relieve light or moderate pain. Although often described in the literature as a COX-2 inhibitor, meloxicam is significantly less selective of this enzyme than traditional COX-2 inhibitor drugs. Meloxicam is chemically designated as 4-hydroxy-2-methyl-N-(5-methyl-2-thiazolyl)-2H-1,2-benzothiazine-3-carboxamide-1,1-dioxide and its molecular weight is 351.4. Its empirical formula is C₁₄H₁₃N₃O₄S₂ and its structural formula is shown in FIG. 1. Meloxicam is a solid, practically insoluble in water, with higher solubility observed in strong acids and bases. It is very slightly soluble in methanol. Meloxicam has an apparent partition coefficient of (log P)app=0.1 in n-octanol/buffer pH 7.4. Meloxicam has pKa values of 1.1 and 4.2.

Mechanism of Action: Like other nonsteroidal anti-inflammatory drugs, meloxicam inhibits COX-1 and COX-2 (cyclooxygenase) enzymes. These enzymes catalyze the conversion of arachidonic acid to prostaglandin G2, which is the precursor for other prostaglandins and thromboxane A2. COX-2 favors the synthesis of prostaglandins that have a physiological function on gastric mucosa and renal activity.

Meloxicam is more selective of COX-2 than indometacin, but less selective than selective inhibitors of the enzyme such as celecoxib or robecoxib. It does not inhibit platelet aggregation induced by collagen or arachidonic acid but it does significantly reduce the production of thromboxane in platelets.

Pharmacokinetics: Meloxicam is quickly absorbed (89%) almost immediately after administration (orally). Absorption is not altered by food intake. Maximum concentration is achieved within 4 to 11 hours after administration. Its half-life ranges from 15 to 20 hours and since a second maximum concentration peak occurs around 12-14 hours after administration, meloxicam undergoes enterohepatic circulation.

Meloxicam is 99.5% bound to plasma proteins and its volume of distribution is 13 to 16 Liters. It is metabolized in the liver through oxidation by CYP3A4 to four inactive metabolites. Its major metabolite is formed by the action of cytochrome P450 and especially isoenzyme CYP3A4.

Meloxicam excretion is in the form of metabolites with 43% excreted in the urine while the remainder is excreted in the feces. Total clearance ranges from 0.4 to 0.5 L/hour. As a result, meloxicam displays linear pharmacokinetics and attains a steady state after 5 days of dosage. Women exhibit lower plasma concentrations relative to men of the same age.

Carisoprodol: Carisoprodol is a skeletal muscle relaxant which is chemically related to meprobamate. This medication has proved to be effective in treating secondary spasms of skeletal muscle.

Carisoprodol was analyzed in 1980 (Elenbass, J. K. Centrally acting oral skeletal muscle relaxants. Am J. Hosp. Pharm., 1980 37:1313-1323) using subjective assessments and a suitable design, which did not permit the superiority of any of the muscle relaxants to be established. However, what was apparent was the decreased effectiveness on chronic problems compared to acute ones. Its formula is shown in FIG. 1.

Mechanism of Action: Not precisely known. This drug does preferentially depress polysynaptic reflexes and at high doses, monosynaptic reflexes (Elenbass, 1980), however sedation is important particularly in high doses.

Pharmacokinetics: Carisoprodol is properly absorbed in the gastrointestinal tract and reaches maximum concentration within 2 to 4 hours. However, the initial musculoskeletal relaxant effect is observed between 30-45 minutes.

Carisoprodol is widely distributed and metabolized in the liver to meprobamate and to hydroxymeprobamate by the CYP 2C 19 enzyme. It is primarily excreted in the urine in the form of meprobamate. Its elimination half-life is 8 hours.

Carisoprodol is not recommended for pregnant or breast-feeding patients. In addition, it is not recommended for patients operating machinery.

### OBJECTS OF THE INVENTION

The general aim of the present invention is to provide a new composition for oral administration, which contains a COX-2 inhibitor drug and a muscle relaxant. Based on the composition of the pharmaceutical form, the COX-2 inhibitor is able to be released at approximately the same speed as the relaxant, both in less than an hour.

In particular, this invention refers to a new use for a COX-2 inhibitor such as meloxicam and for a muscle relaxant such as carisoprodol. When acting together, these two drugs produce a synergic or boosting effect, which is neither obvious nor derivable from the prior art. For the patient requiring such treatment, this effect is more effective in controlling pain than when these components are used separately.

In addition, the present invention refers to a new use for a COX-2 inhibitor such as meloxicam and the carisoprodol. When the two drugs act jointly, the inventors found a greater decrease of known side-effects than when the two components are administered separately to the patient.

Another objective of the present invention is to provide a pharmaceutical composition wherein, after oral administration, both drugs (meloxicam and carisoprodol) are released into aqueous media almost immediately, particularly in less than 1 hour.

Moreover, the new pharmaceutical composition comes in the form of tablets, of proven stability, unit dose, and which contain two drugs: carisoprodol, in a quantity of 200 to 600 mg, and meloxicam in a quantity of 7.5 to 15 mg, both of which are released simultaneously and immediately.

Another objective of the present invention is to provide a pharmaceutical composition in tablet form which can be administered orally, preferably in one or two doses per day, according to the patient's requirements.

An additional objective of the present invention is to provide a pharmaceutical composition that combines two active principles and which also contains at least one of the following excipients: polyvidone K 30, which functions both as an agglutinating and solubilizing agent; crospovidone, which acts as a desintegrant; sodium citrate which acts as a pH regulator; colloidal silicon dioxide, which acts as a glidant; sodium lauryl sulfate, which acts as a solubilizing agent; microcrystalline cellulose PH 101, which acts as a diluent along with lactose monohydrate; and magnesium stearate, which acts as an anti-adherent. The product can also be created with or without purified water.

Another objective of the present invention is to provide a pharmaceutical composition that includes one or more of the following active drugs: a) a COX-2 inhibitor, where the COX-2 inhibitor was selected from a group consisting of rofecoxib, celecoxib, flosulide, meloxicam, nabumetone, etodolac, and nimesulid; b) a muscle relaxant selected from a group consisting of alcuronium, alosetron, aminophylline, baclofen, carisoprodol, and methocarbamol.

### DETAILED DESCRIPTION OF THE INVENTION

The present invention provides a pharmaceutical composition useful in the treatment of muscular or skeletal problems such as torticollis; twists; sprains; dislocations; sports and accidental traumas; muscle, tendon and ligament distensions; painful syndromes of the vertebral column (lumbar pain, sciatica, etc.); post-traumatic and post-operative pain; fractures and other ailments accompanied by inflammation and muscle contraction. It can also be used to relieve acute pains of certain types of rheumatic pains (osteoarthritis, arthrosis, extrarticular rheumatism, ankylosing spondylitis, and signs and symptoms of rheumatoid arthritis.)

In holistic therapy, depending on the case, it could be advisable to recommend an alternative treatment such as hydrotherapy, physiotherapy, etc.

The present description refers to a new invention consisting of pharmaceutical forms, of proven stability, unit dose, and in tablet form, which contain two drugs of immediate release. Concentration may vary according to dose, however studies preferentially deal with tablets containing 200 mg of carisoprodol, acting as a muscle relaxant, and with 7.5 mg or 15 mg of meloxicam, which is a type of COX-2 inhibitor.

The present invention has full technical support due to the results observed in the phase II clinical studies which refer to the effectiveness of the designed pharmaceutical forms. This is shown in the proof of concept study comparing the effectiveness of the administration of one dose of meloxicam and carisoprodol simultaneously, in the same formulation, against the placebo and against meloxicam alone, as described in the examples section.

The abovementioned phase II clinical studies, (see examples further on), depict the results seen in patients with grade I and II sprains. while these patients may recover with traditional recommended treatments such as ice, rest, elevation of the injured limb, and bandaging, the administration of drugs proved to increase the effect of these physical treatments. In addition, these studies found that the administration of carisoprodol-meloxicam in doses of 200 and 15 mg respectively, as shown in the results of Example 5 of the present description (see FIG. 5), produces an important analgesic effect after about 90 minutes from administration (white arrow in FIG. 5). Meloxicam alone produces a delayed analgesic effect, up to 360 minutes after administration (dark arrow in FIG. 5). The combination of carisoprodol-meloxicam was also found to facilitate functional recovery, in other words the ability to lift weight, 30 minutes after administration. This effect contrasts with that of the group that only received meloxicam, whereby the analgesic effect and functional recovery were observed later. It has been noted that the administration of carisoprodol alone may produce analgesia in patients experiencing lower back pain and those experiencing dental pain.

Upon comparing the carisoprodol-meloxicam formulation with meloxicam as a single drug, the phase I clinical study series found a statistically significant decrease in levels of erythrocytes in the feces, hematocrit and hemoglobin, which may indicate that a minor intestinal bleeding event may occur.

From a symptomatological point of view, patients who received the combination reported adverse effects which primarily corresponded to carisoprodol. Unlike results using higher doses of carisoprodol, the tested dose (200 mg) did not produce any changes in the physiological tests suggesting that such a dose is safe and does not alter the subject's reaction time or awareness, allowing the subject to go about his/her normal daily activity. In reference to the decrease in blood pressure which is observed with the single dose of carisoprodol and the carisoprodol-meloxicam combination, this could be a reflex of the anxiolytic activity of carisoprodol. However, although this effect was observed in volunteers who received the drug for 15 days, it is no different from the other treatments.

Overall, it was concluded that the carisoprodol-meloxicam combination in a single formulation is safe for short-term administration (15 days), does not cause intestinal damage, and (unlike other formulations on the market) does not produce any decrease in response time or any decrease in attention and concentration.

In general, the characteristics of the drugs are very different and this poses a problem during manufacturing since it is difficult to obtain a product whose drug contents are uniform.

The present invention proposes a new stable pharmaceutical composition designed to contain combinations of two active principles in amounts which allow for the control of muscular or skeletal problems though one or two oral administration per day. The pharmaceutical composition contains the two biologically active agents in a single phase and in a homogeneous composition. The two agents are substantially different from one another in their inherent physicochemical properties and are required in significantly disproportionate proportions in regard to concentration, weight and volume.

The active principle of the new pharmaceutical composition is meloxicam, a derivative of benzothiazine carboxamide, which in turn is a derivative of enolic acid. Meloxicam is combined with a second active principle, carisoprodol, a skeletal muscle relaxant which is chemically related to meprobamate. This drug has proved to be effective in treating secondary ailments of musculoskeletal contractions and as an adjunct to rest after physical therapy.

As described in detail and despite the differences in proportions and physicochemical properties, this invention presents a composition whose stability is pharmaceutically acceptable. During storage there is no possibility that the two drugs will degrade and during release no problems of incompatibility have been observed which delay the release of either of the drugs, according to the desired parameters, even when different concentrations are used (7.5 to 15 mg of Meloxicam) and (200 to 600 mg of Carisoprodol). Both drugs of the invention's pharmaceutical composition dissolve quickly and are released in less than one hour.

In dissolution tests carried out on the product, the release is similar to that of commercial products containing only one of the drugs, in other words tablets containing only meloxicam and tablets containing only carisoprodol. For both drugs, release was around 95% respectively (see Table 2). According to this data, it can be deduced that due to its composition, the tablet of the present invention allows for immediate release of both drugs whereby the two drugs do not compete for greater dissolution.

In addition, this invention also describes the process of obtaining the abovementioned composition. From a technical point of view, this composition has an acceptable level of laboriousness or difficulty during industrial scale manufacturing, which translates into production cost benefits.

The procedure for manufacturing the granulate consist in mixing the components of the formula carisoprodol, polyvidone K 30, 50% of crospovidone, 50% of microcrystalline cellulose PH 101, lactose monohydrate, sodium lauryl sulfate and 75% of colloidal silicon dioxide, all this mixture is screened to break lumps.

The obtained product in the previous step is mixed in a Collette Ultima granulation machine for 5 to 20 minutes using the main mixer at a speed of 50-150 rpm and the chopper at a speed of 300-600 rpm. The powder mixture is then moistened with purified water under the following operating conditions: 5 minutes using the main mixer at a speed of 70-120 rpm and the chopper at a speed of 300-700 rpm. Liquid is added to granulate at an average flow rate of 7 to 15 mL/sec. The thermal balance system of the granulation bowl and lid is kept at 30° C. The powders are granulated under the following operating conditions: 5 to 10 minutes using the main mixer at a speed of 120 rpm and using the chopper at a speed of 900 rpm. The thermal balance system of the granulation bowl and lid is kept at 30° C.

The obtained granulate is dried in progressive stages under the following operating conditions: 5 to 10 minutes at a speed of 20 to 65 rpm using the main mixer and at a speed of 900 rpm using the chopper. The vacuum system is kept at a value below 80 mbar inside the granulation bowl and the thermal balance system keeps the lid at 60° C. The abovementioned conditions are maintained for 10 minutes and afterwards the microwave system is activated to maintain operation at 50% of capacity (0.5 KW). In the 20 minutes that follow, the operating conditions of the equipment are changed. All conditions are kept the same with the exception of the microwave system which is changed to 75% of its capacity (0.75 KW). The swinging bowl system allows the granulation bowl to be positioned at an angle of 45° at its highest point.

Once the above phase has been completed, for 25 minutes the equipment conditions are switched to 100% of microwave capacity and the conditions of the rest of the parameters are kept the same. This procedure continues until granulate moisture levels reach 2.0-3.0 percent.

### Procedure for Obtaining the Tablets

Meloxicam, 25% of Colloidal silicon dioxide, 50% of Crospovidone, and 50% of Microcrystalline cellulose PH 101 are placed in the Gallay Systems Laboratory mixer and mixed for 5 minutes at 12 rpm. This mixture is then milled through a 2A062R caliber screen using a Quadro Comil mill at a drive motor speed of 1200 rpm.

Next, the milled granulate is placed in the Gallay mixer along with the carisoprodol granulate mixture and mixed for no less than 25 minutes at 12 rpm.

Then, the magnesium stearate is separately milled through the 2A062R caliber screen using the Quadro Comil mill at a motor drive speed of 1200 rpm. The milled powder of the previous step is then added and they are mixed for 5 minutes at 12 rpm.

The mixture of powders are pressed to specifications in 17.0×7.2 mm dies using a GEA Courtoy Modul tablet press.

The tablets can be packaged in different primary containers, blister packs (PVC, PVDC, PET) and bottles (PEAD, glass, PET), which are subjected to stability studies under accelerated conditions following the international guidelines established in the USP (United States Pharmacopeia 28-National Formulary 23. Rockville, Md.: U.S. Pharmacopeial Convention 2005) and the ICH (International Conference of Harmonization, Guideline for Industry, Stability Testing of New Drug Substances and Products. ICH-Q IA September 1994).

For the invention described herein, all optional modifications that a person with expertise in the field of the invention may select or carry out are considered to fall within the scope of the present invention.

### DESCRIPTION OF FIGURES

FIG. 1: Structural formula of meloxicam and carisoprodol.

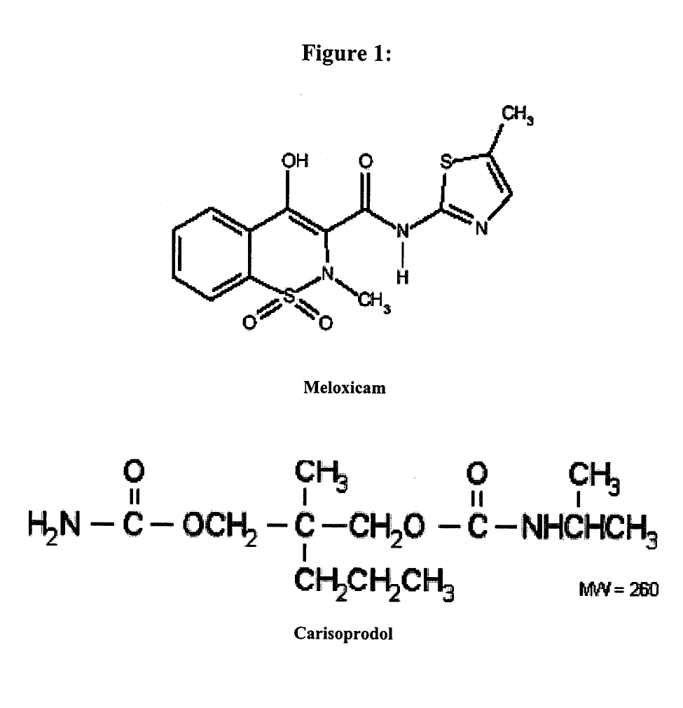

FIG. 2: Qualitative and quantitative formulation of the composition of the invention.

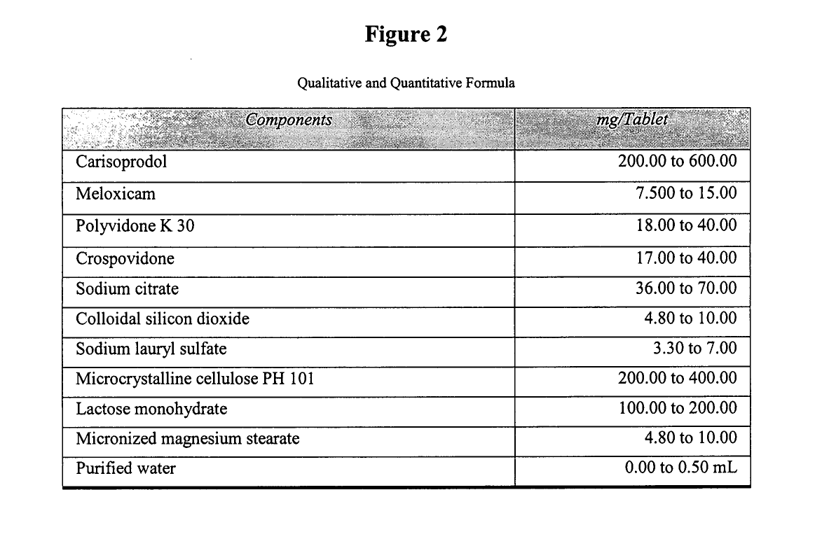

FIG. 3: Description of the final product once the steps mentioned in Example 1 of the present description have been carried out.

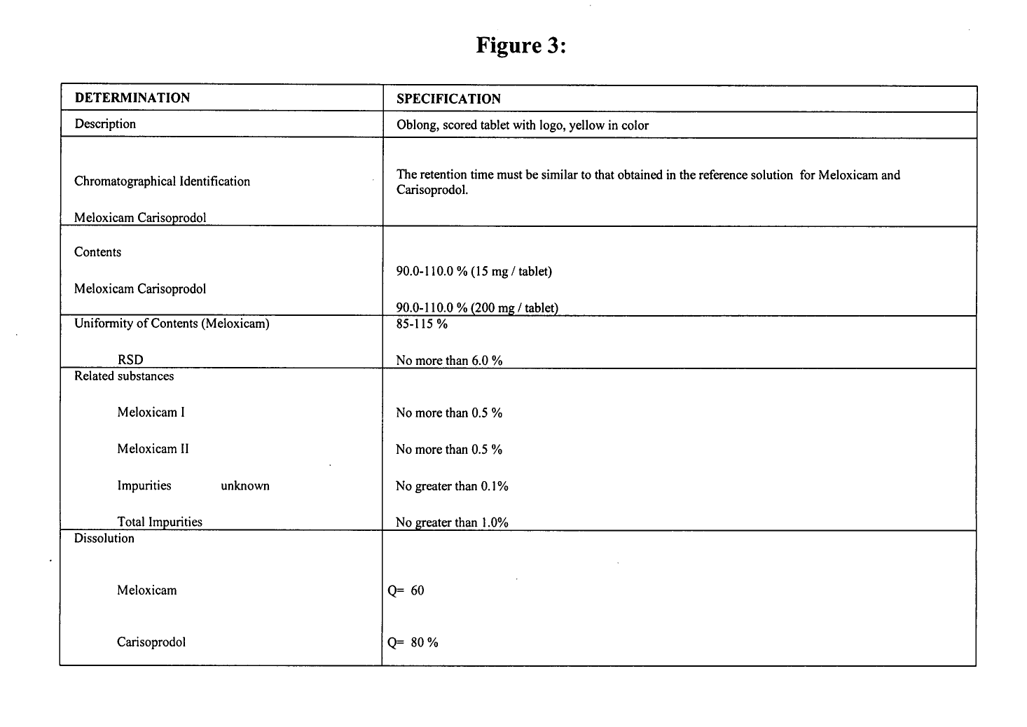

FIG. 4: Outcome of stability studies for the product Dorsal 15/200 mg, tablets.

FIG. 5: Analgesic effectiveness of the treatments. The graph represents the effect-time relationship of three different treatments. Each point represents the response of 10 patients experiencing pain produced by a sprained ankle. As shown, in the case of the carisoprodol-meloxicam combination, the effect is statistically significant when compared to the placebo (p=0.022) as of 90 minutes (white arrow) and the significance increases as 1440 minutes is reached (p=0.0020). Meanwhile, with meloxicam the decrease in the VAS (visual analog scale) is statistically significant as of minute 360 (p=0.021) (dark arrow) and is maintained until minute 1440.

FIG. 6: Analgesic effectiveness of the different treatments. The graph represents the effect-time relationship of three different treatments. Each point represents the response of 10 patients in functional recovery experiencing pain produced by a sprained ankle. As shown, in the case of the carisoprodol-meloxicam combination, the effect is statistically significant when compared to the placebo (p=0.0004) as of 30 minutes (white arrow) and the significance increases as 1440 minutes is reached (p=0.008). Meanwhile, with meloxicam recovery is statistically significant up until minute 1440 (p=0.037) (dark arrow).

### EXAMPLES

The following examples are not meant to be limiting of the scope of the invention, instead, they are merely illustrative and demonstrate the full experimental support which constitutes appropriate information in the technical field of the invention. The examples include a summary for formulating the pharmaceutical composition and a summary for obtaining the tablets. In addition, one example describes the results of the stability studies and the phase I and II clinical studies, which are consistent.

### Example 1

The procedure for obtaining the tablet form of the pharmaceutical composition of the invention, basically consists of the following steps:
a) Mix Carisoprodol, Polyvidone K 30, 50% of Crospovidone, 50% of Cellulose microcrystalline PH 101, Lactose monohydrate, Sodium lauryl sulfate and 75% of colloidal silicon dioxide, and sift using a 15 to 60 sieve.
b) Mix the sifted product obtained in the previous step in the Collette Ultima granulator for 5 to 20 minutes using the chopper at a speed of 300 to 600 rpm.
c) Moisten the powder mixture with purified water in a one-pot granulation machine (the Ultima) at a flow rate of 7 to 15 mL/sec.
d) Dry the obtained granulate in progressive stages in the same machine and process in the chopper for approximately 60 minutes with a lid thermal balance between 50° and 70° C., under pressure conditions below 80 mbar, with nitrogen gas injected into the system and the use of microwaves, according to the recommended indications that are provided in the chopper equipment being used, until moisture levels reach 2.0-3.0 percent.
e) Mix the Meloxicam, 25% of the Colloidal silicon dioxide, 50% of Crospovidone, 50% of Microcrystalline cellulose PH 101 for 5 minutes at 12 rpm, using the Gallay Systems machine.
f) Mill the mixture using a 2A062R caliber screen using a Quadro Comil mill at a drive speed of 1200 rpm.
g) In the Gallay Systems Laboratory unit, mix the previous mixture with the mixture containing the carisoprodol granulate for no less than 25 minutes at 12 rpm.
h) Using the 2A062R caliber screen, separately mill the micronized magnesium stearate using the Quadro Comil mill at a drive speed of 1200 rpm and add the milled powder obtained in the previous step and mix for 5 minutes at 12 rpm.
i) Using a GEA Courtoy Modul press, compress the obtained powder mixture according to the established specifications using 17.0×7.2 mm dies.

The tablets may be stored in different primary containers, blister packs (PVC, PVDC, PET) and bottles (PEAD, glass, PET), which are subjected to stability studies under accelerated conditions following the international guidelines established by the USP (United States Pharmacopeia 28-National Formulary 23. Rockville, Md.: U.S. Pharmacopeial Convention 2005) and the ICH (International Conference of Harmonization, Guideline for Industry, Stability Testing of New Drug Substances and Products. ICH-QIA September 1994).

### Example 2

Presentations formulated for the final product

| Proportion of Meloxicam | Carisoprodol |
|---|---|
| 7.5 mg | 200 mg |
| 15 mg | 200 mg |

### Example 3

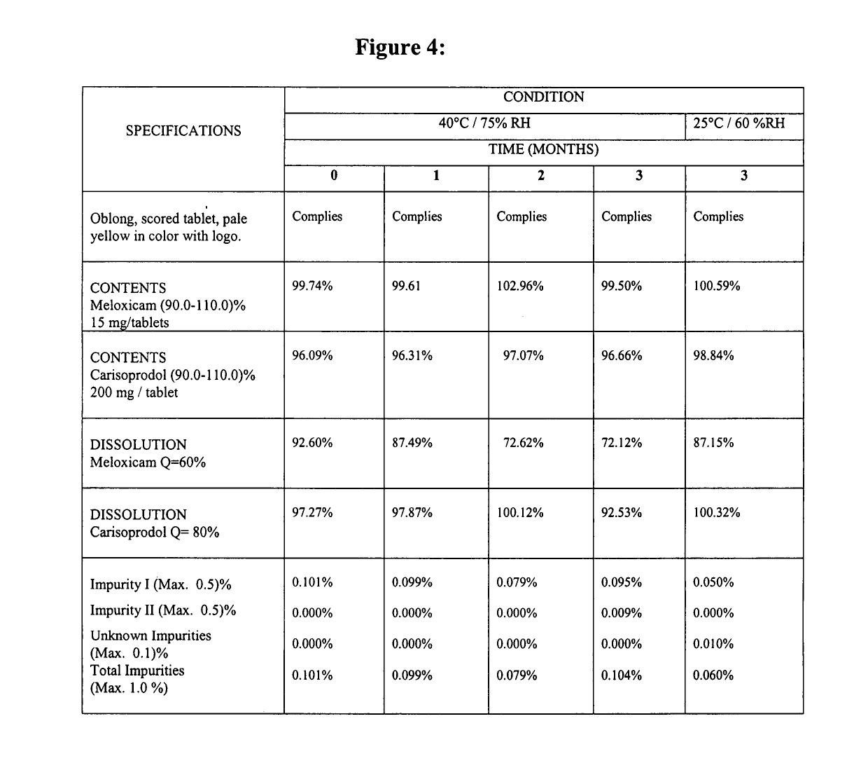

The stability studies conducted on the pharmaceutical compositions manufactured according to the description provided in the present invention were carried out under specific conditions of temperature and humidity: 40° C. and 75% relative humidity and 25° C. and 60% relative humidity. The study was conducted over a period of 3 months and proved that the pharmaceutical composition is stable, as shown in the results in FIG. 4.

### Example 4

A phase I study was conducted on the bioavailability, safety and tolerability of meloxicam and carisoprodol administered in the same formulation and separately. Sixty healthy male volunteers participated in the study and were divided into four treatment groups. The compounds administered were: Treatment A (tablets containing a combination of 200 mg of carisoprodol and 15 mg of meloxicam), Treatment B (tablets containing 15 mg of meloxicam), Treatment C (tablets containing 200 mg of carisoprodol), and Treatment D (placebo). The study was prospective, single-blind, parallel, longitudinal, and comparative with a placebo and with the components of the combination administered separately, with randomized distribution into 4 groups. Bioavailability was studied after the administration of a single dose of the medications in the study and also during the steady state (15 days after medication administration). For meloxicam and carisoprodol, the determination of the pharmacokinetic parameters of Cmax and the area under the plasma concentration curve with regard to time (ABCo-t and ABCo-inf), allow for the characterization of the bioavailability of the drug in its pharmaceutical form, in terms of speed and level of absorption respectively. The application of the statistical tests on data transformed into natural logarithms, determined that no statistically significant differences exist between treatments when evaluating the pharmacokinetics of the initial dose. Meanwhile, the multiple dose scheme at steady state showed an apparent difference in most of the parameters evaluated, where plasma levels attained in Treatment A (meloxicam combined with carisoprodol) were generally lower than those of Treatment B (meloxicam on its own). In the case of single-dose meloxicam, either in combination with carisoprodol or on its own, the quotients of probability that the values would be below or above the permissible limits were always less than 5% (p<0.05). However, when meloxicam was repeatedly administered, the values were above 5%. As a result, it can be concluded that the acute administration of meloxicam, either combined or on its own, is bioequivalent. As for carisoprodol, there is no bioequivalence in any condition. This indicates that the combination of 15 mg of meloxicam and 200 mg of carisoprodol in a formulation has a different bioavailability than the individual components, which also suggests that the combination modifies the bioavailability of its components, or rather that the pharmaceutical aspects influence the amount of drug that is absorbed, especially when administered repeatedly.

Incidentally, in relation to the tolerability and safety studies, the present clinical study indicates that the combination of meloxicam-carisoprodol 15/200 mg is at least as tolerable as meloxicam alone and as carisoprodol alone.

### Example 5

Pharmacokinetic Study of Single Dose: Volunteers received their assigned drug randomly and blood samples were taken at the following intervals: 0, 15, 30, 60 and 90 min, as well as 2, 3, 4, 4.5, 5, 5.5, 6, 8, 12 and 24 hours after drug administration.

Pharmacokinetic Study in Steady State: Type of study: three-arm, parallel, randomized, prospective, longitudinal, single-blind, balanced.
Group A: Received the combination of 200 mg of carisoprodol and 15 mg of meloxicam
Group B: Received 15 mg of meloxicam
Group C: Received 200mg of carisoprodol.

Subjects were administered a daily single dose of the medication for 15 days. A pharmacokinetic study was conducted on day 1 and day 14 so that bioavailability could be clearly identified for single dose and for multiple doses, and so that the bioequivalence of the combined product could be compared to the individual products.

Maximum concentration (Cmax) was found to be similar in the meloxicam-carisoprodol combination and in the 15 mg dose of meloxicam on its own, both for single doses and for maintenance doses (bioequivalence). Gastrointestinal tolerability measured by and endoscope was found. No significant differences were observed in the gastric mucosa upon comparing the meloxicam-carisoprodol combination with the placebo. As for the sedative effect of carisoprodol, no significant differences were observed between the results of the MINIMENTAL test conducted prior to medication administration and the results of the MINIMENTAL test conducted after administration. This occurred when carisoprodol was administered alone in doses of 200 mg and when the meloxicam-carisoprodol combination was administered. As for gastrointestinal bleeding, a statistically significant decrease was found in hematocrit and hemoglobin when meloxicam was administered alone in doses of 15 mg. These changes were not found when the meloxicam-carisoprodol combination was administered.

### Example 6

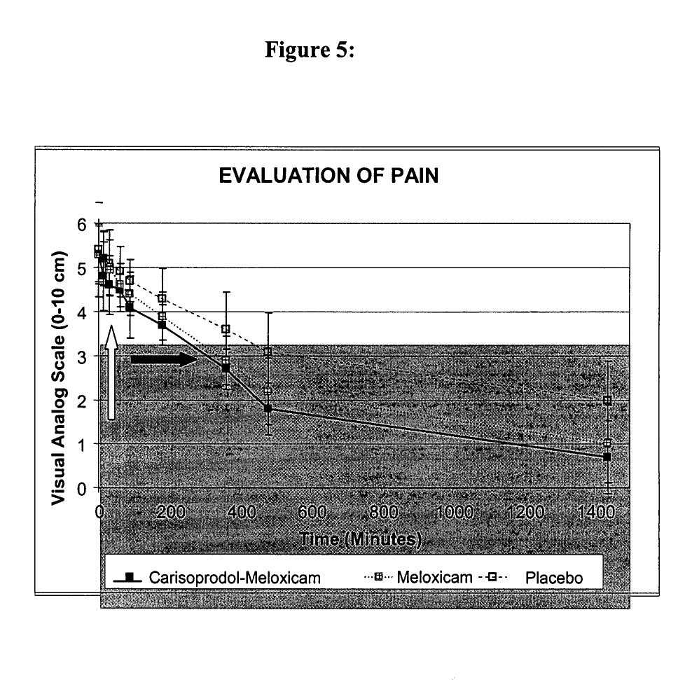

Evaluation of Pain through the Visual Analogue Scale: V.A.S. The study was prospective, single-blind, parallel, longitudinal, with 30 male patients who gave informed consent. While practicing sports, these patients suffered grade I or grade II sprained ankles. Patients were randomly divided into three treatment groups: Treatment A: tablets containing 200 mg of carisoprodol and 15 mg of meloxicam; Treatment B: tablets containing 15 mg of meloxicam; Treatment C: placebo. Treatment using physical measures significantly decreases the pain rating in the VAS scale in patients receiving the placebo, however this effect is not complete. The administration of meloxicam-carisoprodol or meloxicam reduces the rating on the VAS scale in a statistically significant way, with ratings below 1 (see graph in FIG. 5).

### Example 7

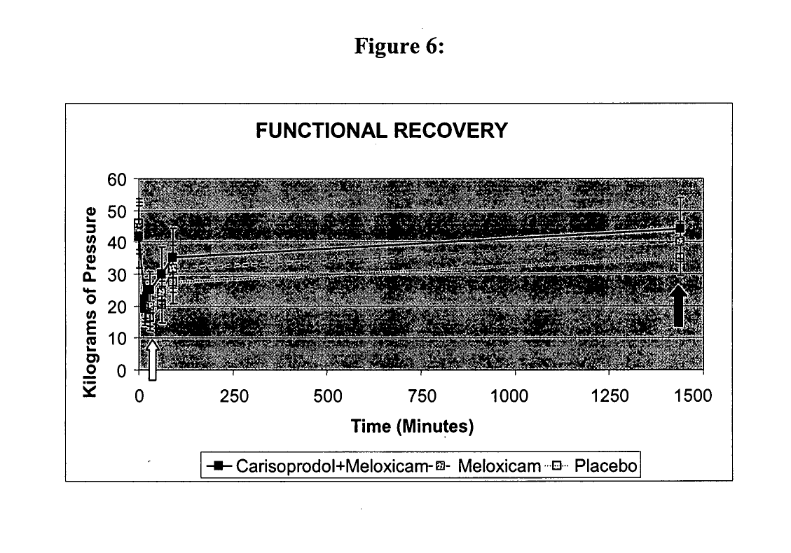

Functional Recovery. In all cases, in the volunteers was observed a functional recovery either in group treated with meloxicam-carisoprodol, or with the meloxicam treatment as wall as the placebo group. In these result have stood out that said recovery occurred faster in the group that received meloxicam-carisoprodol. Statistical comparision of meloxicam-carisoprodol group with placebo group, the difference is evident as from minute 30. When comparing the mexloxicam group placebo group, both are statistically different at 24 hours (see graph in FIG. 6).

Anyone skilled in the art could substitute the components to make the excipient of several formulations, including those suitable for formulating oral suspensions. These components could include, for example: colloidal silicon dioxide, hydroxymethylcellulose, sorbitol, glycerol, xylotol, sodium phosphate monobasic, sodium saccharine, sodium benzoate, citric acid, artificial flavoring, and purified water. Thus, within the framework of the description, the additives, excipients, proportions of the components, methods of formulation and other parameters described herein, can be modified or substituted by a variety of options.

1. A table pharmaceutical composition comprising a COX-2 inhibitor in combination with a muscle relaxant, both of immediate release.

2. The pharmaceutical composition of claim 1, wherein the composition comes in tablet form.

3. The pharmaceutical composition of claim 1, wherein the COX-2 inhibitor is Meloxicam and the muscle relaxant is Carisoprodol.

4. The pharmaceutical composition of claim 2, wherein the COX-2 inhibitor is Meloxicam and the muscle relaxant is Carisoprodol.

5. The pharmaceutical composition of claim 1, wherein the composition contains a quantity equivalent to 7.5 to 15 mg of Meloxicam and a quantity equivalent to 200 to 600 mg of Carisoprodol.

6. The pharmaceutical composition of claim 2, wherein the composition contains a quantity equivalent to 7.5 to 15 mg of Meloxicam and a quantity equivalent to 200 to 600 mg of Carisoprodol.

7. The pharmaceutical composition of claim 3, wherein the composition contains a quantity equivalent to 7.5 to 15 mg of Meloxicam and a quantity equivalent to 200 to 600 mg of Carisoprodol.

8. The pharmaceutical composition of claim 4, wherein the composition contains a quantity equivalent to 7.5 to 15 mg of Meloxicam and a quantity equivalent to 200 to 600 mg of Carisoprodol.

9. A treatment method that consist of medication to the patient of meloxicam and carisoprodol to relieve muscular or skeletal pain such as torticollis; twists; sprains; dislocations; sports and accidental traumas; muscle, tendon and ligament distensions; painful syndromes of the vertebral column (lumbar pain, sciatica, etc.); post-traumatic and post-operative pain; fractures and other ailments accompanied by inflammation and muscle contraction, sharp pains of certain types of rheumatic pains such as: osteoarthritis, arthrosis, extrarticular rheumatism, ankylosing spondylitis, and signs and symptoms of rheumatoid arthritis.

10. The method of treatment according to claim 9, wherein said medication is a tablet containing 200 mg of carisoprodol and 15 mg of meloxicam, which can be administered in one or two daily doses.

11. The method according to either claim 9, wherein said medication causes a decrease in pain and produces functional recovery as of minute 90 after administration.

12. The method according to either claim 10, wherein said medication causes a decrease in pain and produces functional recovery as of minute 90 after administration.

13. The method according to claim 9, wherein said medication is unlikely to cause the side effect related to bleeding of the digestive system.

14. The method according to claim 10, wherein said medication is unlikely to cause the side effect related to bleeding of the digestive system.
